(19) United States
(12) Patent Application Publication
(10) Pub. No.: US 2009/0156933 A1
(43) Pub. Date: Jun. 18, 2009

(54) ULTRASOUND SYSTEM FOR RELIABLE 3D ASSESSMENT OF RIGHT VENTRICLE OF THE HEART AND METHOD OF DOING THE SAME

(75) Inventors: Olivier Gerard, Viroflay (FR); Pau Soler, Paris (FR); Pascal Allain, Versailles (FR)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/066,094

(22) PCT Filed: Sep. 7, 2006
(86) PCT No.: PCT/IB06/53163

§ 371 Date: Sep. 10, 2008

(30) Foreign Application Priority Data
Sep. 7, 2005 (EP) 05300724.1

(51) Int. Cl. A61B 8/13

(52) U.S. Cl. 600/443

(57) ABSTRACT

The present invention relates to a method and a system for right ventricular 3D quantification by registering and merging or fusing together several (2-5) 3D acquisitions for an extended field of view in 3D to have the right ventricle in one 3D data set.

The present invention relates to a method and a system for a right ventricular 3D quantification based on the registration of several (2-5) 3D ultrasound data sets to build an extended field of view with improved image quality. This data is then used to quantify the right ventricle of the heart, otherwise this is very difficult to have in one dataset due to its complex shape. In particular, the present invention relates to acquiring a full 3D ultrasound image by register and merging or fusing together several (2-5) 3D acquisitions for an extended field of view in 3D to have the right ventricle (RV) in one 3D dataset.

Right ventricular function is currently not well studied in cardiac diseases due to its complex shape and the lack of quantified measures. However, it has become increasingly clear that reliable and reproducible quantified values of the RV volumes are very important and carry important prognosis values.

U.S. Pat. No. 6,780,152B2 to Ustuner, et al. relates to a method and apparatus for ultrasound imaging of the heart. However, this patent relates to 2D (2 dimensional) imaging and does not provide a solution for a 3D image of the RV in one dataset. In fact, this patent has the requirement of being co-planar, which strictly limits its use.

The present invention relates to a method and a system for right ventricular 3D quantification by registering and merging or fusing together several (2-5) 3D acquisitions for an extended field of view in 3D to have the right ventricle in one 3D data set.

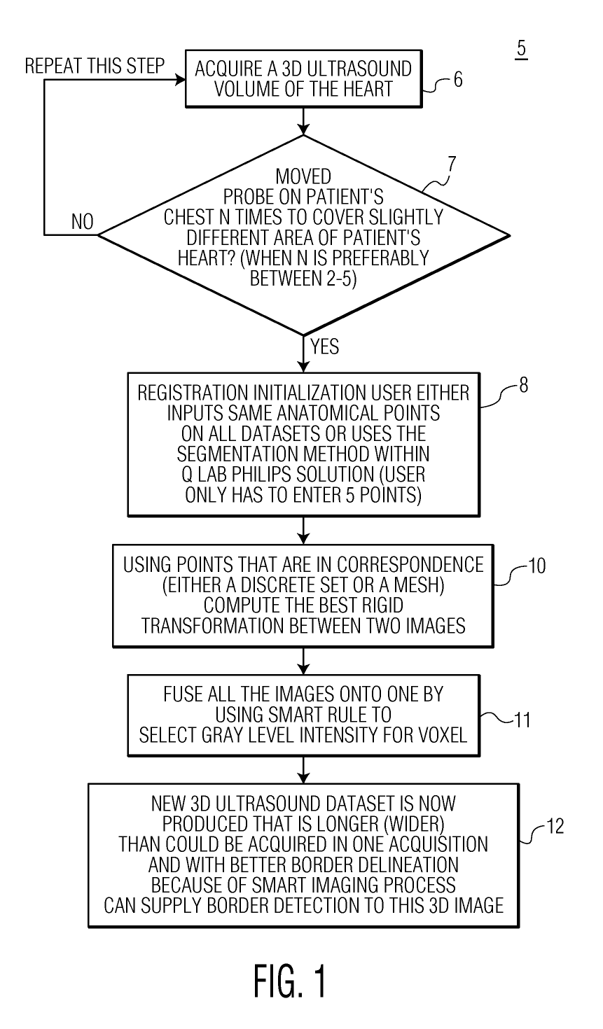

FIG. 1 is a general flow chart of the present invention;

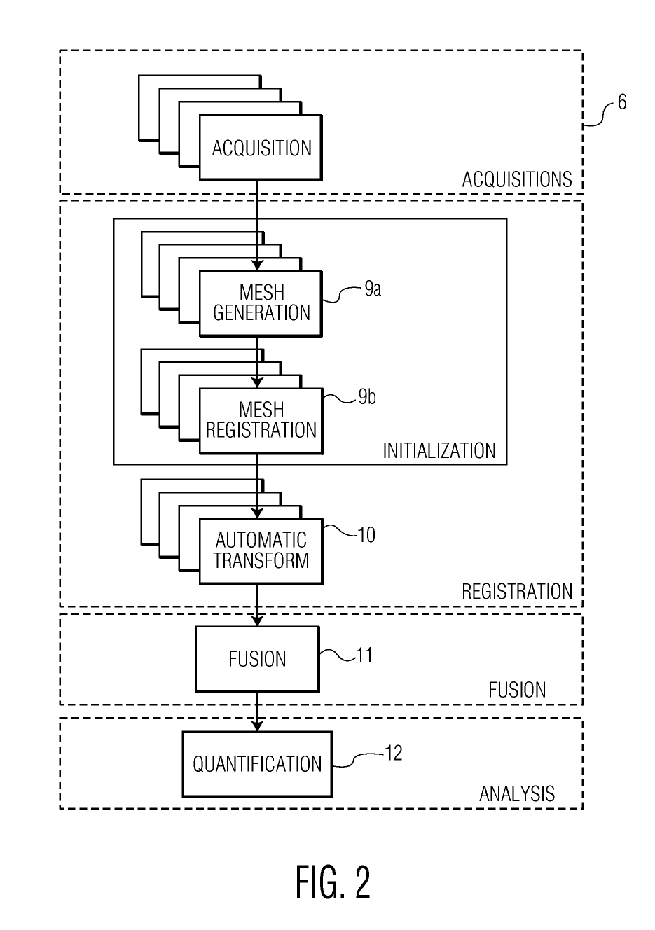

FIG. 2 is a detailed flow chart of a preferred embodiment of steps of FIG. 1;

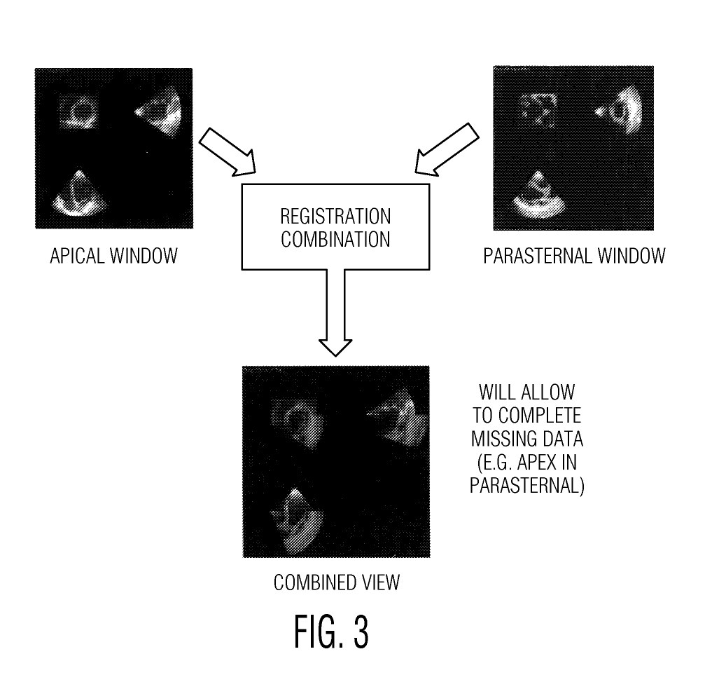

FIGS. 3A-C illustrate a typical 3D ultrasound image registration;

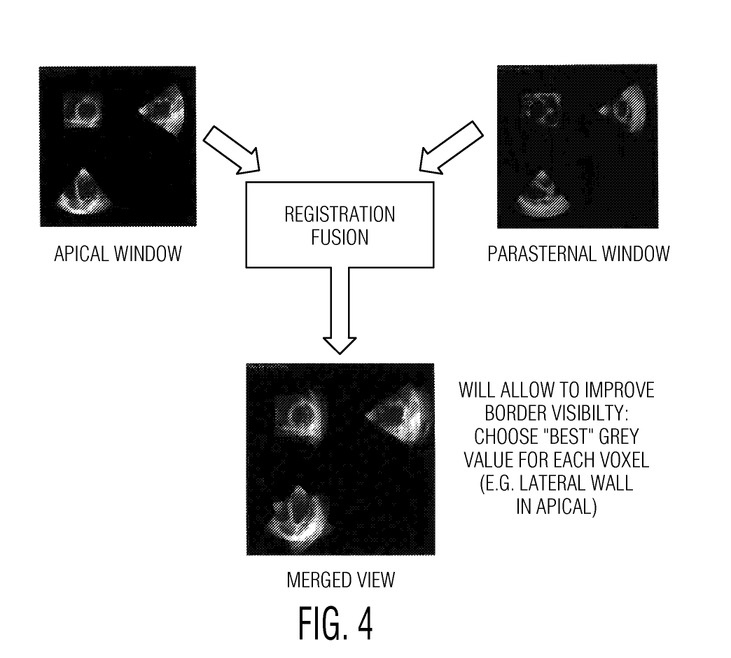

FIGS. 4A-C illustrate the 3D ultrasound image registration with fusion according to the teachings of the present invention;

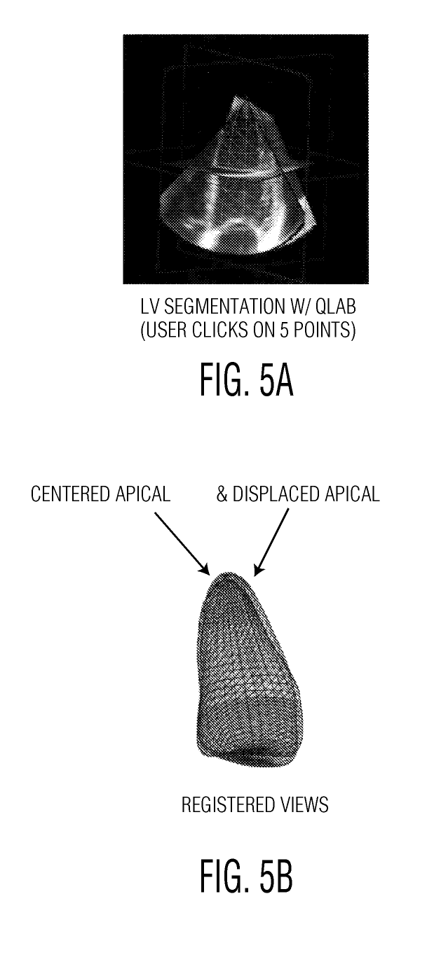

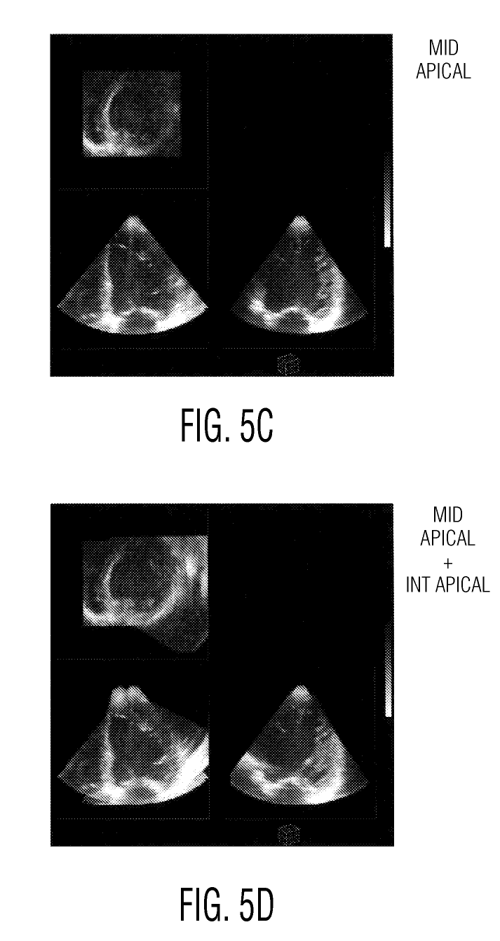

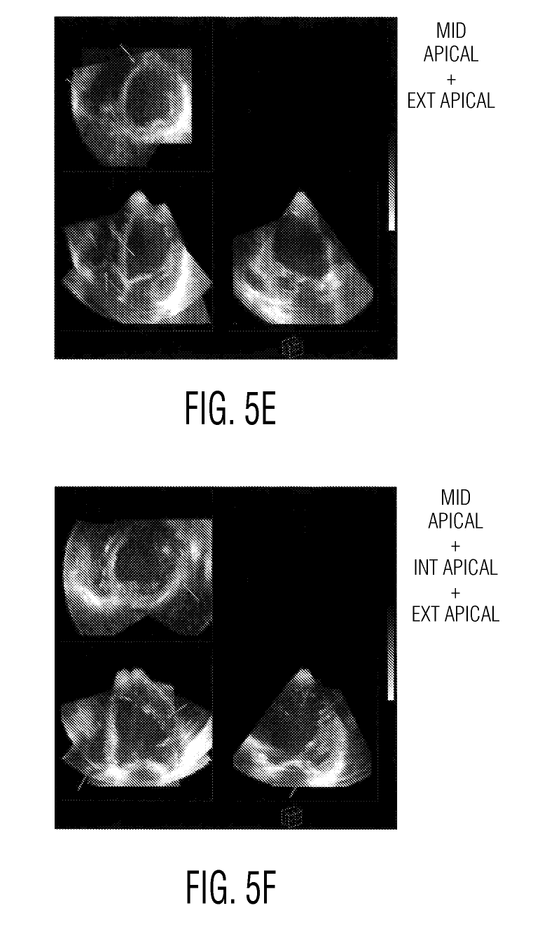

FIGS. 5A-F illustrate images for registration according to the teachings of the present invention; and

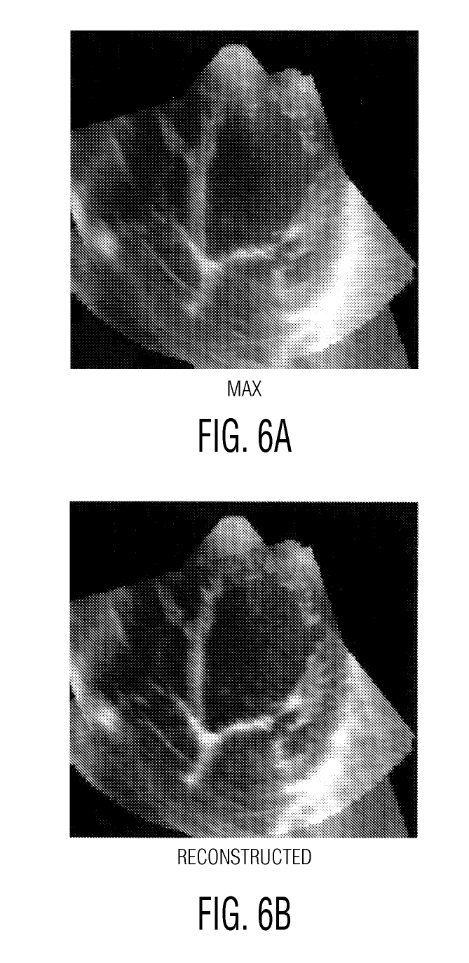

FIGS. 6A-B illustrate the fusion steps of the present invention.

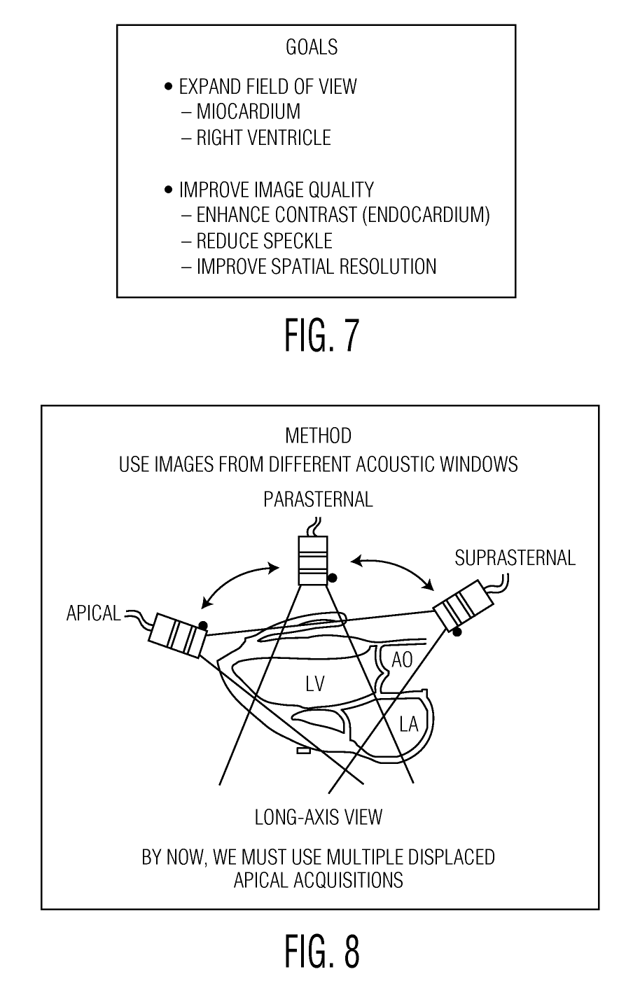

Referring now to FIGS. 1-8, FIG. 1 is a general flow chart 5 of the method and system of the present invention.

First a three dimensional (3D) ultrasound volume of a patient's heart is acquired using known ultrasound equipment such as, but not limited to, Philips' Sonos 7500 Live 3D or EE 33 with the 3D option or with a 3D echograph from the GE vivid 7 Dimension apparatus. Any 3D acquisition will do for step 6.

An ultrasound probe is then moved slightly on a patient's chest preferably 1 to 2 cm in order to cover a different area of the patient's heart in step 7 of FIG. 1. Step 6 is then repeated so that step 6 is done at least twice and preferably 2-5 times. If step 6 is performed n times, preferably 2≦n≦5, is done then there are n acquisitions and n datasets into which the anatomical points need to be inputted by the user in step 8, described below. In the acquisition stage, the user acquires several (between 2 and 5) ultrasound data sets, most probably in a full volume mode (maybe with high density). The different views, from different points of view and different insonifying angles provide complimentary data about the heart of the patient.

This completes the acquisition portion of the present invention.

Registration is then initialized (step 8) by either asking the user to provide all the same anatomical points on all data sets acquired in steps 6-7 or else by using the segmentation method provided in the apparatus of Philips' Q-Lab Solution where a user has only to enter 5 points. The Q-Lab solution is discussed in detail below with reference to the embodiment of FIG. 2. The acquired data sets are registered in order to know their relative positions in 3D space. Registration step can be done fully automatically or semi-automatically with the user providing a few points to guide the process.

FIG. 2 describes a preferred embodiment of step 8 of FIG. 1 in which the segmentation method of the Philips Q-Lab Solution is used for inputting points on the datasets acquired by repeating steps 6 and 7 n times.

The acquisition step 6a is shown as was described in steps 6 and 7 of FIG. 1. Registration initialization (step 8 of FIG. 1) is done by mesh registration 9a and mesh registration 9b of FIG. 2. The segmentation method of step 8 of FIG. 1 can be conducted by placing a mesh in a 3D data set—in three steps described below (these 3 steps are already part of Philips' Q-Lab product—the 3D Q Advanced plug in.

Step 1: The user enters 4 or 5 references points on the 3D dataset (typically 3 or 4 mitral value level and one at the endocardial apex).

Step 2: The best affine deformation is then determined between an average LV shape (including the reference points) and the 5 points (by the way of the 5 points which are matched).

Step 3: An automatic deformation procedure is then applied to this average shape to match the information contained in the 3D dataset (typically a 3D “snake-like” approach, well known to the experts in the image processing field).

This procedure leads to a 3D mesh following the LV endocardial border placed in the 3D dataset. It is also significant to note that the usage of the reference points also indicates the orientation of the mesh. It means that each vertex (3D point) of the mesh can be automatically marked (for instance: basal, mid, apical, septum wall, papillary muscle . . . ).

Then this procedure is repeated for all the datasets acquired in step 6 of FIG. 1.

All the resulting meshes are matched together (9b of FIG. 2). More specifically, the best rigid transformation between the meshes and the 1^{st} one are computed. Taking advantage of the anatomical specifics, each vertex has its correspondence in the other meshes. Namely vertex #i in mesh #j should be matched with vertex #i in mesh #k. The best rigid transformation is found by minimizing the sum of the squared error (or any minimization procedure). An example of this mesh registration phase is illustrated in FIGS. 6a and b (before and after mesh registration).

This rigid transformation based on the mesh provides an initialization for the registration procedure.

It is understood that embodiment of FIG. 2 is an illustrative example but is not intended to limit the present invention to this one embodiment.

In the acquisition step of FIG. 2, a user can acquire:

a. A standard apical 3D ultrasound volume of the heart;

b. A displaced apical 3D ultrasound volume moving the U/S probe on the patient chest by about 2 cm to the left from the initial position.

In the registration step of FIG. 2, a user can:

Use the segmentation method already available within QLab Philips solution (user has only to enter 5 points). This process will generate mesh of about 600 points for each acquisition.

Use the correspondence between the points of the meshes, a rigid transformation is computed for each acquisition to the reference acquisition (e.g. standard apical acquisition). Denoting by {pᵢ} the reference point set and by {p′ᵢ} the source point set, the best rigid transformation (which is composed by a rotation matrix R and a translation vector T), in a least-squares sense, is computed as:

$$p = \frac{1}{N} {\sum }_{i} {p}_{i}$$

$${p}^{′} = \frac{1}{N} {\sum }_{i} {p}_{i}^{′}$$

$${q}_{i} = {p}_{i} - p$$

$${q}_{i}^{′} = {p}_{i}^{′} - {p}^{′}$$

$$R = argmin {\sum }_{i} {q}_{i}^{″} {Rq}_{i}$$

$$T = {p}^{′} - Rp$$

where R can be obtained with a singular value decomposition (SVD) method.

During the fusion step of FIG. 2, a user can fuse all the images onto one by using smart rule to select grey level intensity for each voxel. In fact, the fusion is performed via the multichannel deconvolution operation described below. This is the smart rule—a software procedure performed on the central unit of the echograph (suitable equipment by way of example but not limiting the present invention thereto include Philip's Sonos 7500, iE33 or any other equipment capable of acquiring 3D data)—the smart rule is a multichannel deconvolution method described as follows: The highest quality is obtained by using a multichannel Deconvolution method. By denoting each of the acquired volumes as vᵢ, the fused volume v is obtained as:

$$v = argmin [ {\sum }_{i} {‖ {v}_{i} - {h}_{i} * v ‖}^{2} + λΨ ( v ) ]$$

where v can be obtained using the conjugate gradient methods, hi is the point spread function of each acquisition, Ψ represents a regularization operator (e.g. Tikhonov Ψ=∥Δv∥²) and λ represents the degree of regularization.

In this way, the user has a new 3D ultrasound data set that is:

larger (wider) than could be acquired in acquisition;

with better border delineation, because of the smart merging process.

One can then apply border detection on this new image that could not be applied before, for instance right ventricle detection (because it is difficult to have fully the RV in one single acquisition) and complete heart detection with left and right ventricles.

Each step functionality could be implemented in different ways. Some of the feasible alternatives are listed as follows:

### Acquisition

Use other displacements within apical window. (use only standard U/S equipment (echograph) by placing only the U/S probe at different positions on the patient's chest.)

Use other acoustic windows than apical, in particular parasternal and subcostal. (use only standard U/S equipment (echograph) by placing only the U/S probe at different positions on the patient's chest.)

### Registration

Initialize by user selected landmarks. Typically, these are points of anatomical importance that are easily located in all acquisitions. Indeed, this favors the matching of structures that might be of special interest for the user. (use software in Philip's Qlab).

Use a geometrical transformation with higher number of freedom degrees, in particular affine or elastic transformations. (use software in Philip's Qlab).

Alternatively, a position tracker (e.g. magnetic, optical) can be attached to the probe to provide the relative positioning of the different acquisitions. (Use an external piece of equipment with two parts: one attached to the U/S probe and another piece of equipment to detect and track the position of the first part eg. the probe. By way of example but without limiting the present invention thereto this second piece of equipment for detecting and tracking the probe can include localizer technologies for both optical and electromagnetic detection and tracking of the probe provided by Northern Digital, Inc. These parts are commercially available and can rely on the electromagnetic or optical localization method.

Fusion (this step is software only and the software is in the Philip's Qlab).

Use wavelet-based fusion rules.

Non-linear fusion (e.g. maximum operator)

Adaptive fusion (angular dependent).

FIGS. 3A-3C illustrate a type of 3D ultrasound image registration. FIG. 3A is an image of an apical window and FIG. 3B is an image of a parasternal window. FIG. 3C shows the image as a combined view with registration.

As noted previously, segmentation-based registration can serve as a starting point. Some of the issues involved included sensitivity to user clicks, difficult in displaced apical segmentation and variability with (one) cardiac cycle among views.

Alternatively, automatic registration has some issues as well, namely a need to improve robustness of the image, noisy data and partial coverage.

FIGS. 4A-4c show the advantages in the present invention over FIGS. 3A-3C with registration and for according to the present invention. FIG. A again shows an apical window image and FIG. 4B shows a parastemal window that are merged by registration and fusion into the combined view image of FIG. 4C. The fused image will allow the user to improve border visibility by choosing the best gray value for each voxel (e.g. lateral well in apical region).

1. An ultrasound method for reliable 3D assessment of a right ventricle of a patient's heart, the steps comprising;
acquiring a 3D ultrasound volume of a patient's heart;
moving a 2D matrix ultrasonic probe to a slightly different area of said patient's heart and repeating step (a) until step is done n times where 2≦n≦5 before going on to step (c);
initialization of registration of n images acquired from steps (a) and (b) wherein anatomical points are input to all datasets;
computing a best rigid transformation between n images acquired from steps (a) and (b) by using said anatomical points in each of said n images that are in correspondence;
fusing said n images onto one image by using smart rule to select gray level intensity for Voxel; and
applying border detection to 3D image obtained by the fusing step (e) so that a new 3D ultrasound dataset is obtained that is longer (wider) than could be acquired in one acquisition and with better border delineation because of smart imaging process of a right ventricle of said patient's heart.

2. The method according to claim 1 where during said initialization of registration step (c) a user inputs same anatomical points on each dataset for 3D ultrasound image acquired for each slightly different area of a patient's heart that is probed.

3. The method according to claim 1 wherein during said initialization of registration step (c) a segmentation method with a Q-Lab Philips Solution is used so a user has to enter five anatomical points.

4. The method according to claim 1 wherein said anatomical points in correspondence in said computing step (d) are a discrete set.

5. The method according to claim 1 wherein said anatomical points in correspondence in computing step (d) are in a mesh.

6. An ultrasound system for reliable 3D assessment of a right ventricle of a patient's heart, comprising;
ultrasonic imaging equipment for acquiring a 3D ultrasound volume of a patient's heart;
a 2D matrix ultrasonic probe adapted to be moved to a slightly different area of said patient's heart and repeating imaging with said ultrasound equipment until it is done n times where 2≦n≦5;
registration controls on said ultrasound equipment for initializing registration of said n images acquired wherein anatomical points are input to all datasets by said controls;
said ultrasound equipment including computing apparatus for computing a best rigid transformation between said n images acquired by using said anatomical points in each of said n images that are in correspondence;
controls on said ultrasound equipment for fusing said n images onto one image by using smart rule algorithm in said ultrasound equipment to select gray level intensity for voxel; and
said ultrasound equipment including border detection controls for applying border detection to 3D image obtained by said fusing so that a new 3D ultrasound dataset is obtained that is longer (wider) than could be acquired in one acquisition and with better border delineation because of smart imaging process of a right ventricle of said patient's heart.

7. The system according to claim 6 where during said initialization of registration a user inputs same anatomical points on each dataset for 3D ultrasound image acquired for each slightly different area of a patient's heart that is probed.

8. The system according to claim 6 wherein during said initialization of registration step (c) a segmentation method with a Q-Lab Philips Solution is used so a user has to enter five anatomical points.

9. The system according to claim 6 wherein said anatomical points in correspondence in said computing are a discrete set.

10. The system according to claim 6 wherein said anatomical points in correspondence in computing are in a mesh.
